(19) United States
(12) Patent Application Publication
(10) Pub. No.: US 2026/0003016 A1
(43) Pub. Date: Jan. 1, 2026

(54) MAGNET DETECTOR FOR FOREIGN BODY INGESTIONS AND A METHOD THEREOF

(71) Applicant: THE JOHNS HOPKINS UNIVERSITY, Baltimore, MD (US)

(72) Inventors: Kenneth NG, Ellicott City, MD (US); Zhenyi Jack YUE, Baltimore, MD (US); Jessica SU, Bayside, NY (US); Xingjian Gavin LU, Baltimore, MD (US); Leela S. GOWLAND, Baltimore, MD (US)

(21) Appl. No.: 19/133,375

(22) PCT Filed: Nov. 30, 2023
(86) PCT No.: PCT/US2023/081809

§ 371 Date: May 28, 2025

Related U.S. Application Data
(60) Provisional application No. 63/385,711, filed on Dec. 1, 2022.

(51) Int. Cl. G01R 33/09; A61B 6/12; G01R 33/00; G01R 33/02; G06F 13/42

(52) U.S. Cl. CPC G01R 33/091; A61B 6/12; G01R 33/0094; G01R 33/0206; G06F 13/4282

(57) ABSTRACT

A system for detecting a magnet is disclosed. The system includes an array of magnetometers; one or more inter-integrated circuit (I2C) multiplexers electrically connected to the array of magnetometers; and one or more wireless transceivers electrically connected to the one or more I2C multiplexers, wherein the array of magnetometers detects a change in electrical resistance caused by a nearby magnetic object by measuring a strength of a magnetic field in three dimensions. Then taking the data from the heatmap and overlaying the data on an x-ray to provide results for the clinician.

### CROSS REFERENCE TO RELATED APPLICATIONS

This application is the national stage entry of International Patent Application No. PCT/US2023/081809, filed on Nov. 30, 2023, and published as WO 2024/118911 A1 on Jun. 6, 2024, which claims the benefit of U.S. Provisional Patent Application No. 63/385,711, filed on Dec. 1, 2022, which are hereby incorporated by reference in their entireties.

### FIELD

This application generally relates to a magnet detector for foreign body ingestion and method thereof.

### BACKGROUND

In recent years, federal regulatory changes have led to the re-emergence of powerful rare earth magnets, which in turn have increased the number of foreign body ingestions of magnets within the pediatric population. If a child consumes a magnet, or worse, several magnets, there is a great risk of serious internal injuries. Foreign body ingestion occur frequently among children and young adults, with 74,725 cases per year. After a series of court decisions overturned a regulation placed by the U.S. Consumer Product Safety Commission which limited the strength and size of magnets in toy sets, there was an observed 444% increase of magnet ingestion calls since 2018. The ingestion of magnets can be life-threatening and can lead to bowel perforation, fistula formation, and even death. The current standard of care for all foreign body ingestion cases is to first classify the swallowed object using the patient's history, obtain an abdominal X-ray and then recommend treatment based on this information. Based on over two dozen clinician interviews at Johns Hopkins and surrounding hospitals in the DMV region, it was anecdotally found that patient history can be unreliable and it is difficult to deduce whether an object is a magnet from only the x-ray scan. Although x-ray scans can show the object's radiopacity and its two-dimensional shape, magnets come in many different shapes and sizes, making it difficult to definitively deduce the presence of a magnet or a harmless metal object (FIG. 1). This uncertainty can delay diagnosis and extend hospital stays, as doctors may need to take multiple x-rays to track and determine whether an object will safely pass or would require surgical or endoscopic removal. Complications such as perforation and fistulae are more likely to occur in children with delayed presentation. Due to the severity of these cases, some doctors will elect to remove the object if they even remotely suspect that it might be a magnet. Therefore, emergency room doctors need a more reliable method to quickly detect the presence of magnets in pediatric patients and improve diagnostic accuracy. There is currently no product on the market that fulfills this need. This disclosure outlines the development of a device that addresses the current gap in the diagnosis of magnetic foreign body ingestions by providing an accurate and consistent data-driven method of detecting magnets that requires minimal training for physicians to use.

However, current technologies are still lacking in magnet detection capabilities.

### SUMMARY

According to examples of the present disclosure, a system for detecting a magnet is disclosed. The system comprises an array of magnetometers; one or more inter-integrated circuit (I2C) multiplexers electrically connected to the array of magnetometers; and one or more wireless transceivers electrically connected to the one or more I2C multiplexers, wherein the array of magnetometers detects a change in electrical resistance caused by a nearby magnetic object by measuring a strength of a magnetic field in three dimensions.

Various additional features can be included in the system including one or more of the following features. Each magnetometer of the array of magnetometers is connected to a I2C multiplexer of the one or more I2C multiplexers. The one or more I2C multiplexers are electrically connected to an I2C bus of the one or more wireless transceivers. The system can further comprise a computer, wherein the one or more wireless transceivers transmit magnetic field data to the computer. The one or more wireless transceivers are electrically connected to the computer by a message queuing broker hosted on the computer. The message queuing broker is a message queuing telemetry transport broker. The computer is programed to localize the nearby magnetic object using both a magnetic heatmap and an x-ray. The computer is programmed to localize the nearby magnetic object by registering a magnetic heatmap image and an x-ray image to create an overlay of the magnetic heatmap image and the x-ray image that allows a clinician to identify magnetic objects that appear radiopaque on the x-ray image. The registration of the magnetic heatmap image and the x-ray image comprises of performing an affine transformation between a plurality of magnetic markers in both the magnetic heatmap image and the x-ray image. The computer is programmed to generate an overlay of a magnetic heatmap and an x-ray image. The computer is programmed to generate a user interface for manipulating images of a magnetic heatmap, an x-ray image, or an overlay of the magnetic heatmap and the x-ray image.

According to examples of the present disclosure, a method for detecting a magnet is disclosed. The method comprises obtaining a magnetic heatmap image and a x-ray image from a target region of a patient, wherein both the magnetic heatmap image and the x-ray image are obtained with a plurality of magnetic markers positioned on the patient; registering the magnetic heatmap image and the x-ray image using the plurality of magnetic markers; creating an overlay of the magnetic heatmap image and the x-ray image based on the registering; and determining a presence to the magnet using the overlay.

Various additional features can be included in the method for detecting the magnet including one or more of the following features. The registering the magnetic heatmap image and the x-ray image comprises performing an affine transformation between the plurality of magnetic markers in both the magnetic heatmap image and the x-ray image. The method further comprises generating a user interface for displaying the magnetic heat map image and the overlay of the magnetic heatmap image and the x-ray image.

According to examples of the present disclosure, a system for detecting a magnet is disclosed. The system comprises a handheld device comprising an array of magnetometers, one or more inter-integrated circuit (I2C) multiplexers electrically connected to the array of magnetometers, one or more wireless transceivers electrically connected to the one or more I2C multiplexers, wherein the array of magnetometers detects a change in electrical resistance caused by a nearby magnetic object by measuring a strength of a magnetic field in three dimensions; and a computer programmed to generate a user interface for displaying a magnetic heat map image, an x-ray image, and an overlay of the magnetic heatmap image and the x-ray image, wherein the one or more wireless transceivers transmit magnetic field data to the computer.

Various additional features can be included in the system including one or more of the following features. Each magnetometer of the array of magnetometers is connected to a I2C multiplexer of the one or more I2C multiplexers. The one or more I2C multiplexers are electrically connected to an I2C bus of the one or more wireless transceivers. The one or more wireless transceivers are electrically connected to the computer by a message queuing broker hosted on the computer. The message queuing broker is a message queuing telemetry transport broker.

### BRIEF DESCRIPTION OF THE FIGURES

FIG. 1 shows X-ray of different ingested objects.

FIG. 2 shows a circuit schematic of a 2×4 sensor array block according to examples of the present disclosure.

FIG. 3A, FIG. 3B, and FIG. 3C show a current prototype (FIG. 3A) Blueprint of Computer Aided Design (CAD) model, showing housing of the iterated housing prototypes, different compartments are shown to demonstrate capability to integrate with various types of sensors (FIG. 3B), and the sensor array laser-cut model of the previously described CAD model (FIG. 3C and FIG. 3D) according to examples of the present disclosure.

FIG. 4 shows a sample of UI, including: three image panels corresponding to Magnes heat map, X-ray result, and overlay; four user-controlled actions to upload each result, generate the overlay, and save the final output image according to examples of the present disclosure.

FIG. 5 shows a heatmap of three key scenarios: a non-magnetic metallic object, a magnetic object, and 2 magnetic objects versus a camera image according to examples of the present disclosure.

FIG. 6 shows a method for magnet detection according to examples of the present disclosure.

FIG. 7 shows an example computing system according to examples of the present disclosure.

FIG. 8 shows a system according to examples of the present disclosure.

FIG. 9 shows another top view of FIG. 8.

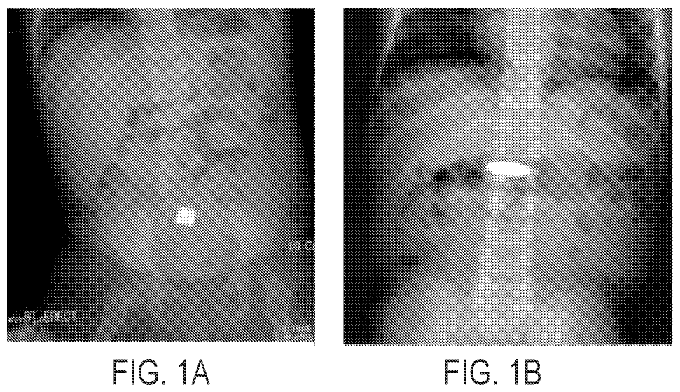

### DETAILED DESCRIPTION

Generally speaking, examples of the present disclosure describe an ergonomic, simple, and reliable device was designed to directly provide clinicians with information regarding the presence of ingested magnets. Through testing with blind scanning, human tissue, and commercially available meat products, it was demonstrated that the device could successfully detect magnets at a variety of ranges accurately and quickly. Furthermore, the device is capable of localization, which allows for the introduction of a method of registering a magnetic field heatmap with x-ray to precisely determine the magnetic properties of radiopaque objects. The magnetic field heatmap represents the magnetic field strength generated by one or more magnets. Examples of the present disclosure provides for a stationary magnet detector that can integrate with X-ray imaging, which is capable of assessing if there is an ingested magnet inside of a patient which would save clinicians valuable time and cost to diagnose the patient using multiple magnetometers for detecting the presence of a magnetic field. These magnetometers create a voltage difference proportional to the strength of the magnetic field, and this detection process will be performed using a computer attached to the device. The method of detection will use real-time data that comes from the magnetometer (magnetic field strength in the x, y, and z directions) which can then be used to calculate the overall magnitude of the magnetic field. The output from the magnet detector would then result in a heat map which would verify the presence if a magnet is present, and be overlayed on the X-ray. This will help pinpoint the exact location of the magnet and can also be used in conjunction with an x-ray scan. Through the use of both technologies, our device would be able to identify the property of the foreign object, and the overlay could determine its exact location.

This product is ground-breaking relative to other available products currently on the market in that with its sensors and calibration method it is designed specifically for medical use and the clinical workflow. The only products that exist on the market today are traditional magnet detectors for commercial use. These are not sensitive enough for a clinical setting to accurately determine the magnetic status of an ingested foreign body with the interference from other equipment and magnetic materials commonly found within hospitals and other clinical settings.

The ingestion of magnets specifically can result in life-threatening injuries and even death. For example, if multiple magnets are ingested, or if one magnet is ingested with a metal object, the magnetic attraction through the intestinal walls can cause internal damage such as bowel perforations and not pass through the body. However, current radiological methods used during the diagnostic workflow for foreign body ingestions are unable to detect the magnetic properties of a foreign body. Without a detection method for magnetic objects, clinicians rely on the shape of the object on the X-ray scan and prior experience to determine whether surgical or endoscopic intervention is necessary.

There is a clinical need for a method of detecting ingested magnets to accompany the current radiological imaging to improve diagnostic accuracy and reduce the number of unnecessary interventions. According to examples of the present disclosure, a device for detecting ingested magnets can be incorporated into a stationary or mobile system that is easily integrated with the current standard workflow using x-rays.

Children, unconscious patients, and individuals with speech or mental disabilities are often unable to describe the object that they have swallowed which makes it dangerous for the patient if undiagnosed or concludes with an unnecessary surgery if the ingested object was harmless. Our invention would be able to provide the best course of action for the patient and give clinicians a clearer picture of what the patient has ingested.

The disclosed device, system and method provide an improvement over other available products currently on the market in that with its sensors and calibration method it is designed specifically for medical use and the clinical workflow. The only products that exist on the market today are traditional magnet detectors for commercial use. These are not sensitive enough for a clinical setting to accurately determine the magnetic status of an ingested foreign body with the interference from other equipment and magnetic materials commonly found within hospitals and other clinical settings.

An example set of design requirements was decided on based on discussions with numerous stakeholders, including emergency room physicians, pediatric gastroenterologists, and surgeons from more than five hospitals and urgent care centers in the United States. The example criteria below are considered for the device to instantiate a meaningful advancement beyond the current standard of care. The device should not generate a magnetic field strong enough to move an ingested magnetic foreign object within the body. The device returns a clear heatmap with registration markers and any magnetic objects in a short amount of time to promptly guide management. Testing of the device showed that the device can output a result within 30 seconds. The device is intuitive to use. From the clinician interviews, it was found that clinicians preferred to spend a maximum of 10 minutes on learning how to use a new device.

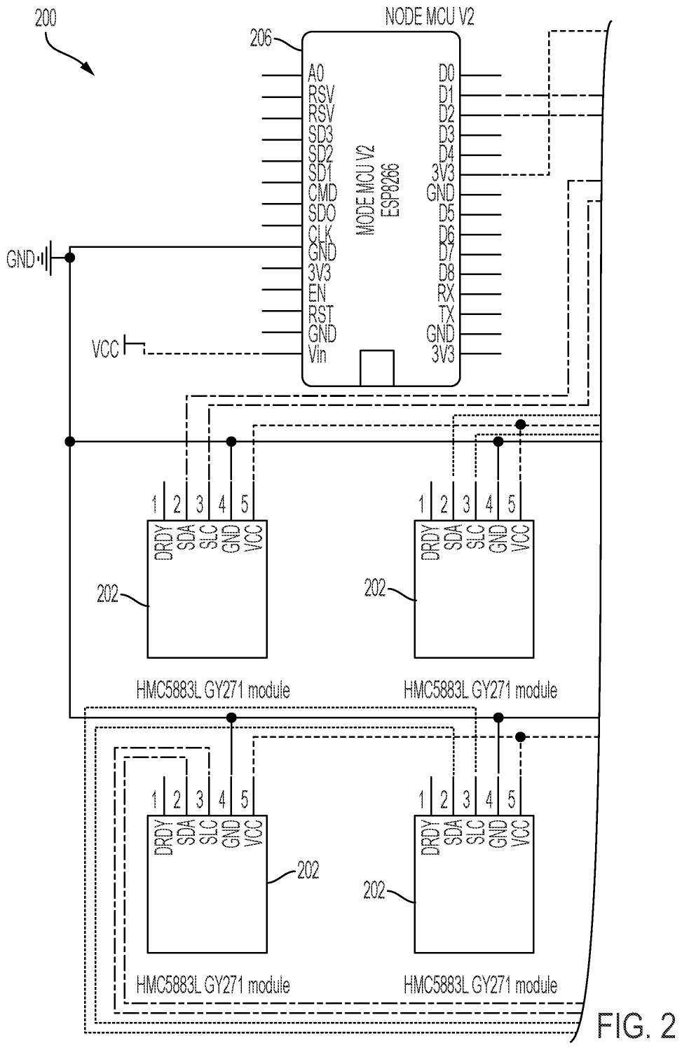

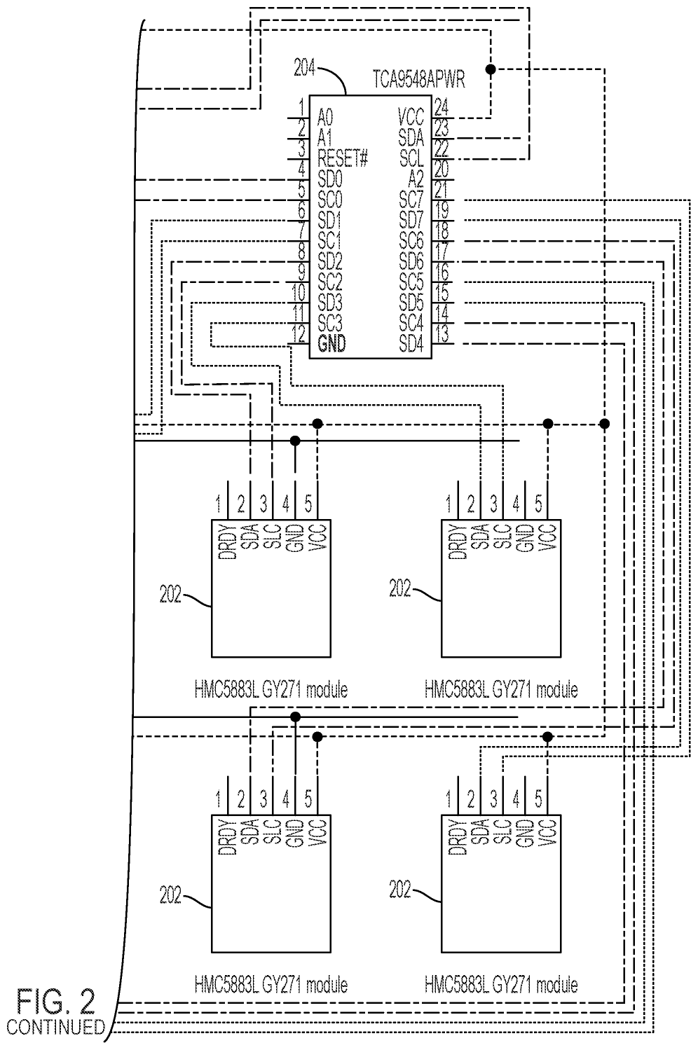

FIG. 2 shows an example circuit schematic 200 of a 2×4 sensor array block of the device according to examples of the present disclosure. The device comprises array of magnetometers 202, for example GY-271 HMC5883L 3-axis magnetic electronic compass module, or other similar type devices. Array of magnetometers 202 are designed for low-field magnetic sensing with a digital interface and operate by converting any magnetic field to a differential voltage output on 3 axes. The voltage shift is the raw digital output value, which is used to determine magnetic fields coming from different directions. Array of magnetometers 202 are connected to inter-integrated circuit (I2C) multiplexers 204, for example TCA9548A, which has eight bidirectional translating switches that can be controlled through the I2C bus, or other similar type of devices, and wireless (WiFi) modules 206, for example NodeMCU ESP8266, which is an open-source platform based on ESP8266 that can connect objects and allow data transfer with the WiFi protocol, or other similar type of devices.

The magnetoresistive magnetometers detect a change in electrical resistance caused by nearby magnetic objects, measuring the strength of the magnetic field (mT) in the x, y, and z directions. Each magnetometer is connected to an I2C Multiplexer, which can connect up to 8 I2C sensors. The multiplexer is then connected to an I2C bus of the Wifi module. The Wifi module transmits the magnetic field data to the computer by connecting to a message queuing telemetry transport (MQTT) broker hosted on a computer. The device itself does not do any processing, and is solely responsible for sending sensor data to the computer for further processing. As such, it is currently powered by a 5V source (e.g., wall outlet or computer's USB port).

The mechanical components of the device as described is mainly a housing for the array of sensors. The exact implementation of the design is not as important as the function of the housing which is to secure the individual sensors in a fixed distance from each other which the computer software can use to extract magnetic field data. The design shown in the image above can be formulated into any size and shape depending on the future development of the device. Generally, as the number of sensors increases over a fixed region, the sensitivity of the device is increased as there are more data points for the system to extract from.

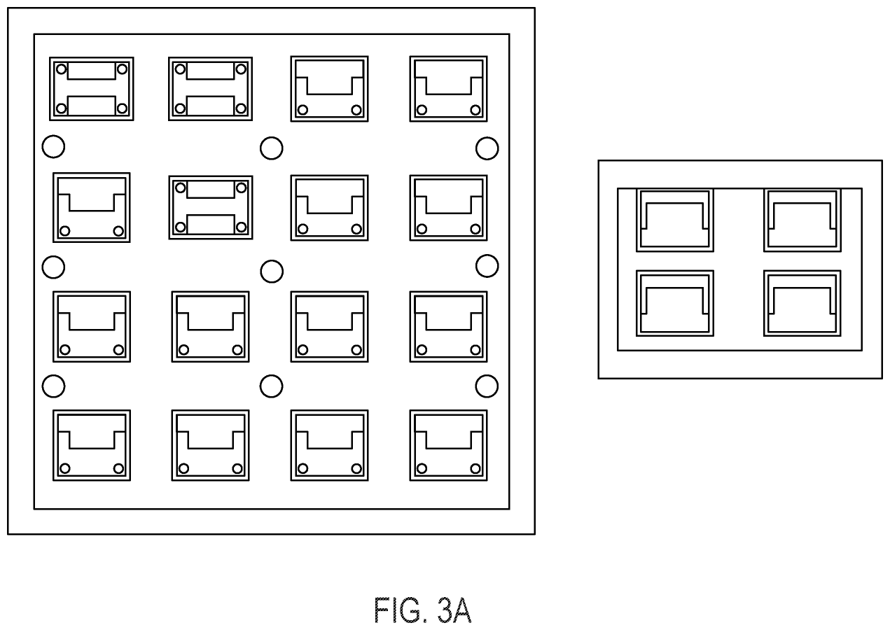

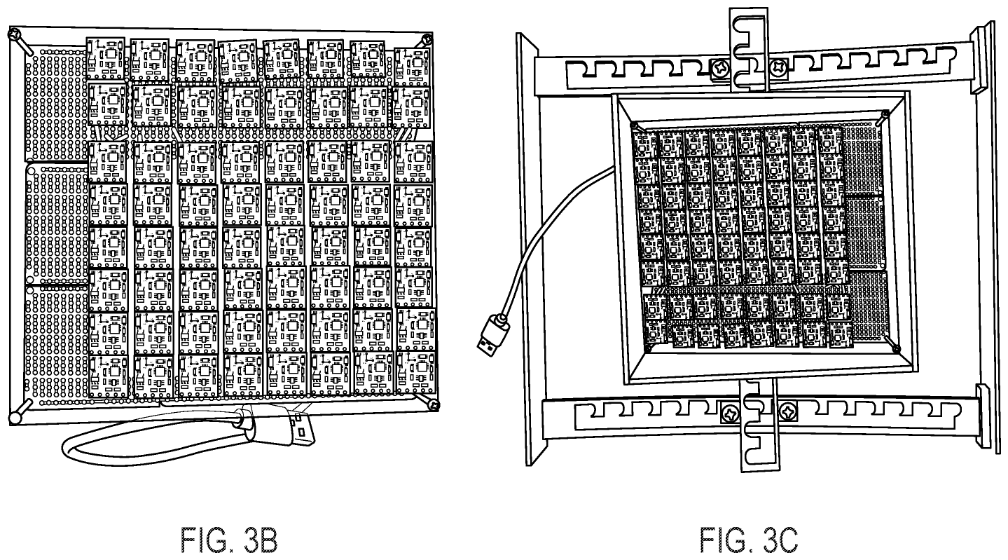

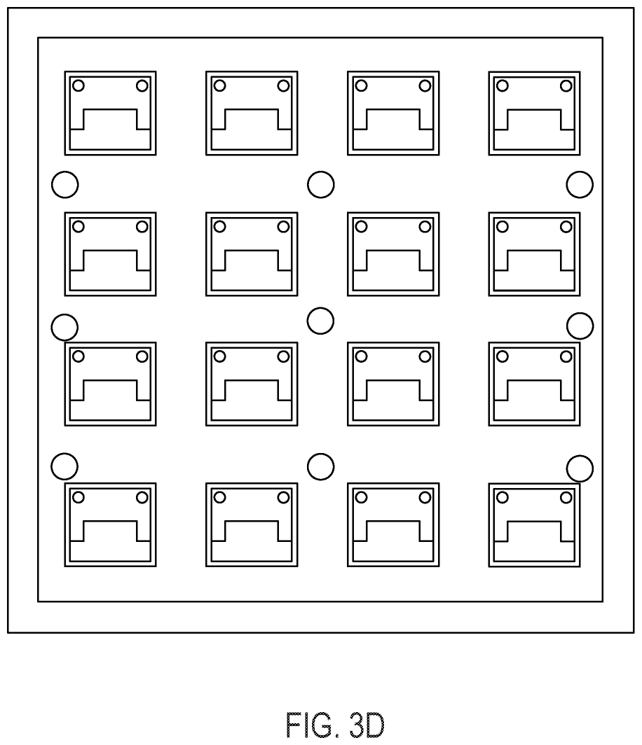

Furthermore, the housing of the device is also able to be moved left/right/up/down on the grooved design on two perpendicular rails. This allows the device to be moved accordingly to the position of the patient and held stationary during the scan (FIG. 3A, FIG. 3B, FIG. 3C, and FIG. 3D).

Examples of the present disclosure provide for methods, devices, and system the ability to localize the magnet on both imaging modalities, the heatmap and X-Ray, and register the 2 images to create an overlay, allowing the clinician to identify magnetic objects that appear radiopaque on the X-Ray. To register the 2 images, 3 very weak magnetic markers are placed on the patient during both imaging procedures. These magnetic markers can be placed in any configuration away from the digestive tract, such that it is not symmetric about the patient's sagittal plane. For example, two markers on each shoulder and the remaining marker at least 2 inches below the shoulder. With these 3 markers appearing on both images, an affine transformation can be found between the 2 sets of points, which allows for the heatmap to be properly overlayed onto the X-Ray.

For example, with these 3 markers appearing on both images, an affine transformation can be found between the 2 sets of points, which allows for the heatmap to be properly overlayed onto the X-Ray. An affine transformation is a linear mapping that preserves points, straight lines, and planes, which is a useful technique to correct for geometric deformations that can occur between taking the x-ray image and the heatmap image. It can be represented using the following set of equations:

$${a}^{′} = {x}_{0} a + {x}_{1} b + {x}_{2}$$

$${b}^{′} = {x}_{3} a + {x}_{4} b + {x}_{5}$$

where (a,b) is mapped to (a′,b′) via the transformation determined by the variables x₀, x₁, x₂, x₃, x₄, x₅. This linear system can then be solved by using the mapping of 3 points, in other words, 2 sets of 3 points.

FIG. 3A, FIG. 3B, FIG. 3C, and FIG. 3D show a current prototype (FIG. 3A) Blueprint of Computer Aided Design (CAD) model, showing housing of the iterated housing prototypes, different compartments are shown to demonstrate capability to integrate with various types of sensors (FIG. 3B), and the sensor array laser-cut model of the previously described CAD model (FIG. 3C and FIG. 3D) according to examples of the present disclosure.

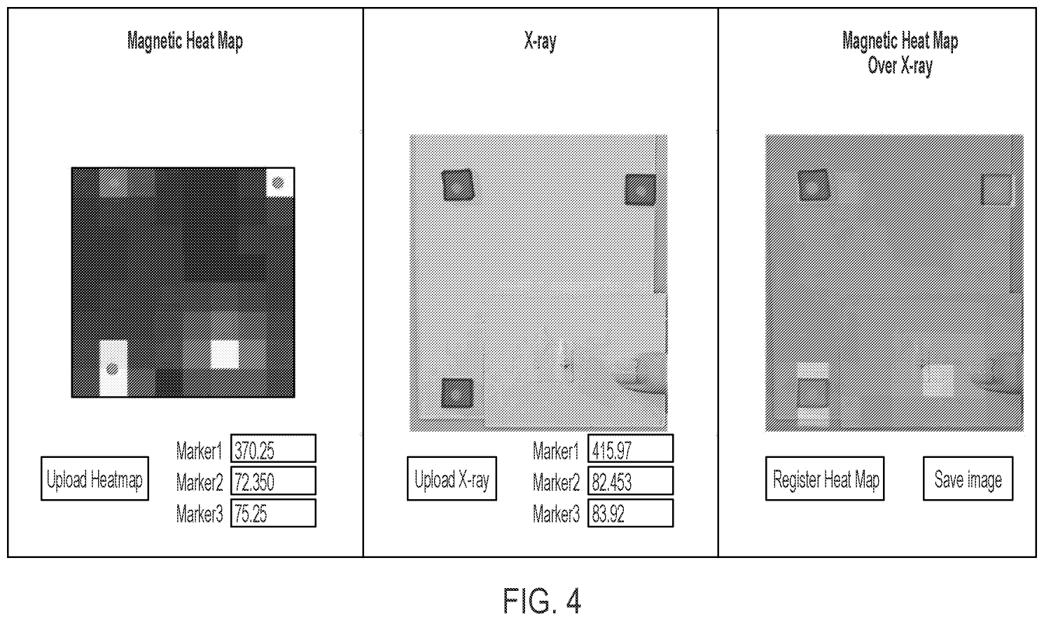

FIG. 4 shows an example user interface (UI) that includes three image panels corresponding to a heat map, an X-ray result, and an overlay, according to examples of the present disclosure. The UI provides for a variety of user-controlled actions, such as the ability to upload the heat map and each X-ray scan result, to generate the overlay using a two or more magnetic markers, and to save the final output image according to examples of the present disclosure. As shown in FIG. 4, the UI provides the ability to combine all the commands for the hardware in one place.

The first test was performed to determine whether our sensors are sensitive enough to detect signal and generate usable data outside of the patient's body. For this purpose, we first searched for the rough size of our target patient group, the pediatric population. We utilized a published measurement of waist circumference in centimeters of people aged 2-18 years to estimate their torso depth, through approximating the transverse plane of a patient's body as an ellipse with a 1:2 ratio between the two axes, and calculating the length of the short axis.

This way, the patient's circumference is approximately 3 pi times of the torso depth. Then, we used the sensor to measure the strength of the magnetic field emitted by a commercially available magnet or another metallic object at several distances, from 0.5 cm to 12 cm. The objects consisted of a standard coin (nickel), a button battery (LR44 1.5V Button Coin Cell Battery, Guangzhou city lichengbei battery Technology Co., Ltd.), a non-magnetic steel ball (Magnetic Sculpture Magnet Building Blocks, HM-CIDIAOSUBALL—LiKee), 6 mm, 8 mm, 10 mm Nickel magnets (Nickel Brushed Refrigerator Magnets, GBYMIUY), a Neodymium square magnet (Magnet Square—0.25″, COM-08643-Sparkfun Electronics), a Neodymium disc magnet (Powerful N52 Rare Earth Magnets—1.26 inch×⅛ inch, 12P-LOVIMAG), a 6 mm Ferrite magnet (Ferrite Refrigerators Magnet 6×2, XXCT-0803-35—Deryun), and a buckyball (Buckyballs Magnets 216 pcs, BB216—Magpole Technology Co. Ltd.). At each distance for each object, 5 scanning trials were performed.

To test the feasibility and utility of the heatmap, 3 representative scenarios were tested—a nonmagnetic radiopaque object, a magnetic radiopaque object and 2 magnetic radiopaque objects. Instead of an x-ray, an image was taken of the setup, which is representative of what might be seen on an x-ray since all of the objects are radiopaque.

To test the feasibility of the heatmap to X-Ray registration, heatmaps were generated based on each test scenario, and a camera image was used to replace the X-Ray image in this new pipeline.

We found that our sensors reliably detect magnets within at least 12 centimeters of the magnet. By the aforementioned calculation, this distance is greater than the torso depth of the vast majority of pediatric patients. We can cover at least 90% for patients 0-18 years old, and at least 95% for patients 0-14 years old.

TABLE 1
Here we show the larger torso depths of older pediatric patients
with an excerpt from published measurement. All reported
waist circumferences from 2-14 years old converted to torso
depth below 12 centimeters. A few outliers may exist, but
our device can still cover the vast majority of pediatric
patients with a detection range of at least 12 centimeters.

| | 90^{th} percentile | 95^{th} percentile |
|---|---|---|
| Age | torso depth | torso depth |
| Group | (cm) | (cm) |
| <14 | <10.9 | <11 |
| 14 | 10.79 | 11.10 |
| 15 | 11.36 | 12.58 |
| 16 | 10.18 | 11.30 |
| 17 | 11.32 | 12.64 |
| 18 | 11.30 | 12.57 |

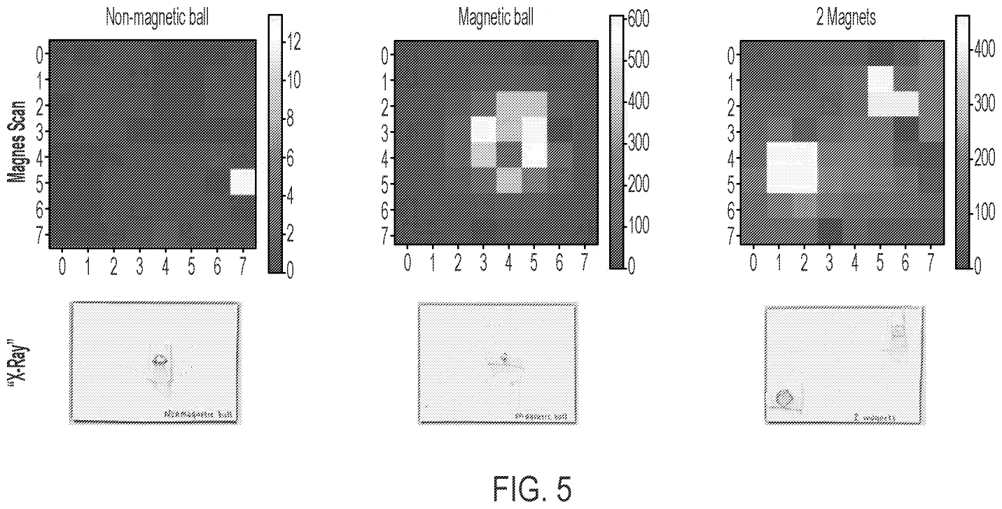

The heatmap generation was successful in all three setups (FIG. 5). With the 2 setups consisting of a single object, the heatmap clearly shows which radiopaque objects are magnetic. This utility is further shown in the scenario with 2 objects, and also shows that the current array has at least enough resolution to distinguish between magnets 2 inches apart.

The heatmap to registration algorithm was successful (FIG. 4). After obtaining the heatmap from the device and getting the X-ray scan, a clinician would manually select the areas corresponding to the weak magnetic markers in each modality. Once the markers have been selected, an overlay of the heatmap onto the X-ray will be generated in the last image, which shows precisely which radiopaque objects are magnetic.

FIG. 5 shows a heatmap of three key scenarios: a non-magnetic metallic object, a magnetic object, and 2 magnetic objects versus a camera image according to examples of the present disclosure.

In summary, examples of the present disclosure provide for a device, a method, and a system that aids clinicians in determining whether a patient has ingested a magnetic object. The experimental results demonstrate the device's ability to distinguish magnetic objects and non-magnetic objects with high accuracy, along with the ability to integrate the resulting magnetic field heatmap with existing X-ray imaging. The results also show that this device is safe to use and will not create complications by generating a magnetic field and moving potential magnets inside the body. Consequently, this device allows clinicians to make diagnoses holistically by combining subjective information (e.g. patient history, and clinician experience) with data.

The device can be further scaled by increasing the number of sensors and taking advantage of PCB fabrication to produce a high resolution and high sensitivity sensor array. Features can also be added to the UI to improve the user experience. For example, adding features to allow the user to interactively click on the image to select the markers, or adjust the amount of overlay of the heatmap onto the X-ray.

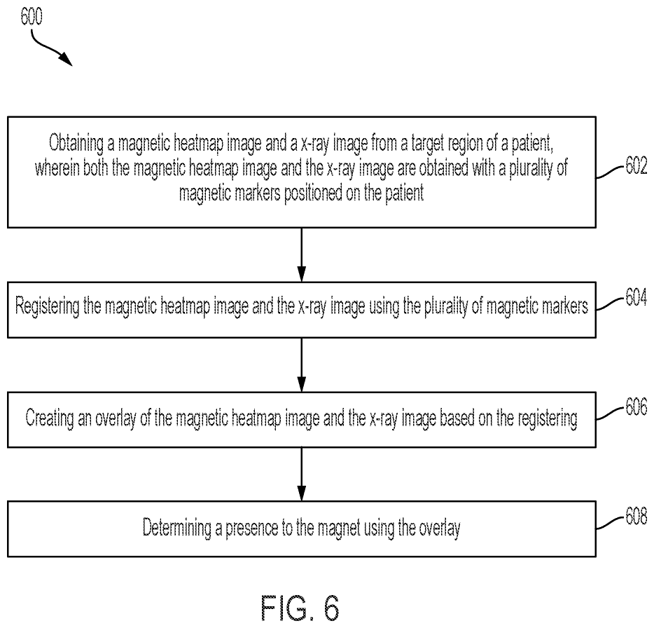

FIG. 6 shows a method for magnet detection 600 according to examples of the present disclosure. In some implementations, one or more process blocks of FIG. 6 may be performed by a computing system 700. As shown in FIG. 6, process 600 may include obtaining a magnetic heatmap image and a x-ray image from a target region of a patient, wherein both the magnetic heatmap image and the x-ray image are obtained with a plurality of magnetic markers positioned on the patient, as in 602. Process 600 may continue by registering the magnetic heatmap image and the x-ray image using the plurality of magnetic markers, as in 604. Process 600 may continue by creating an overlay of the magnetic heatmap image and the x-ray image based on the registering, as in 606. Process 600 may continue by determining a presence to the magnet using the overlay, as in 608.

Process 600 may include additional implementations, such as any single implementation or any combination of implementations described below and/or in connection with one or more other processes described elsewhere herein.

Although FIG. 6 shows example blocks of process 600, in some implementations, process 600 may include additional blocks, fewer blocks, different blocks, or differently arranged blocks than those depicted in FIG. 6. Additionally, or alternatively, two or more of the blocks of process 600 may be performed in parallel.

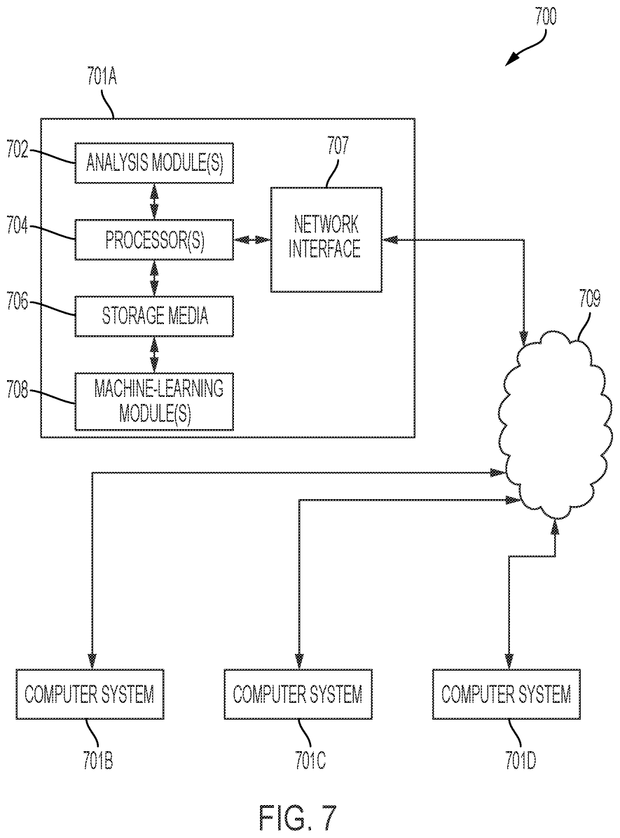

In some embodiments, any of the methods of the present disclosure may be executed by a computing system. FIG. 7 illustrates an example of such a computing system 700, in accordance with some embodiments. The computing system 700 may include a computer or computer system 701A, which may be an individual computer system 701A or an arrangement of distributed computer systems. The computer system 701A includes one or more analysis module(s) 702 configured to perform various tasks according to some embodiments, such as one or more methods disclosed herein. To perform these various tasks, the analysis module 702 executes independently, or in coordination with, one or more processors 704, which is (or are) connected to one or more storage media 706. The processor(s) 704 is (or are) also connected to a network interface 707 to allow the computer system 701A to communicate over a data network 709 with one or more additional computer systems and/or computing systems, such as 701B, 701C, and/or 701D (note that computer systems 701B, 701C and/or 701D may or may not share the same architecture as computer system 701A, and may be located in different physical locations, e.g., computer systems 701A and 701B may be located in a processing facility, while in communication with one or more computer systems such as 701C and/or 701D that are located in one or more data centers, and/or located in varying countries on different continents). A processor can include a microprocessor, microcontroller, processor module or subsystem, programmable integrated circuit, programmable gate array, or another control or computing device.

The storage media 706 can be implemented as one or more computer-readable or machine-readable storage media. The storage media 706 can be connected to or coupled with machine learning module(s) 708. For example, machine learning module(s) 708 can be configured with one or more trained, untrained, or both trained and untrained machine learning algorithms to map one or more heat heaps with the x-rays, as described above, and/or perform additional modeling or computing functions as described above. Additionally, machine learning module(s) 708 can analyze one or more scans to automatically detect and deduce various anomalies. Note that while in the example embodiment of FIG. 7 storage media 706 is depicted as within computer system 701A, in some embodiments, storage media 706 may be distributed within and/or across multiple internal and/or external enclosures of computing system 701A and/or additional computing systems. Storage media 706 may include one or more different forms of memory including semiconductor memory devices such as dynamic or static random access memories (DRAMs or SRAMs), erasable and programmable read-only memories (EPROMs), electrically erasable and programmable read-only memories (EEPROMs) and flash memories, magnetic disks such as fixed, floppy and removable disks, other magnetic media including tape, optical media such as compact disks (CDs) or digital video disks (DVDs), BLURAY® disks, or other types of optical storage, or other types of storage devices. Note that the instructions discussed above can be provided on one computer-readable or machine-readable storage medium, or alternatively, can be provided on multiple computer-readable or machine-readable storage media distributed in a large system having possibly plural nodes. Such computer-readable or machine-readable storage medium or media is (are) considered to be part of an article (or article of manufacture). An article or article of manufacture can refer to any manufactured single component or multiple components. The storage medium or media can be located either in the machine running the machine-readable instructions or located at a remote site from which machine-readable instructions can be downloaded over a network for execution.

It should be appreciated that computing system 700 is only one example of a computing system, and that computing system 700 may have more or fewer components than shown, may combine additional components not depicted in the example embodiment of FIG. 7, and/or computing system 700 may have a different configuration or arrangement of the components depicted in FIG. 7. The various components shown in FIG. 7 may be implemented in hardware, software, or a combination of both hardware and software, including one or more signal processing and/or application specific integrated circuits.

Further, the steps in the processing methods described herein may be implemented by running one or more functional modules in an information processing apparatus such as general purpose processors or application specific chips, such as ASICs, FPGAs, PLDs, or other appropriate devices. These modules, combinations of these modules, and/or their combination with general hardware are all included within the scope of protection of the invention.

Models and/or other interpretation aids may be refined in an iterative fashion; this concept is applicable to embodiments of the present methods discussed herein. This can include use of feedback loops executed on an algorithmic basis, such as at a computing device (e.g., computing system 700, FIG. 7), and/or through manual control by a user who may make determinations regarding whether a given step, action, template, model, or set of curves has become sufficiently accurate for the evaluation of the signal(s) under consideration.

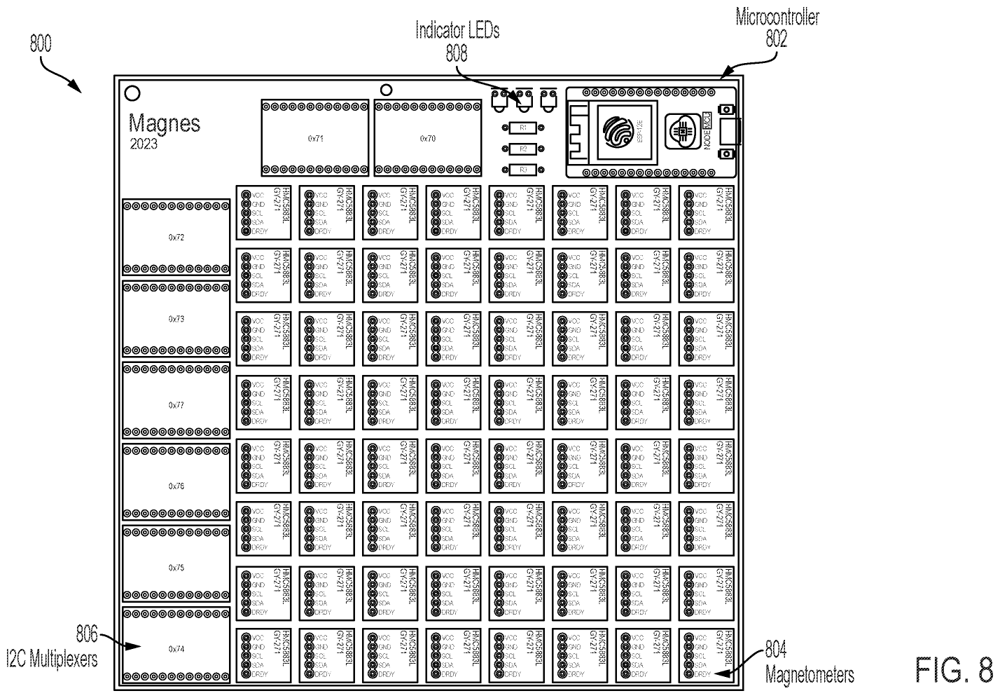

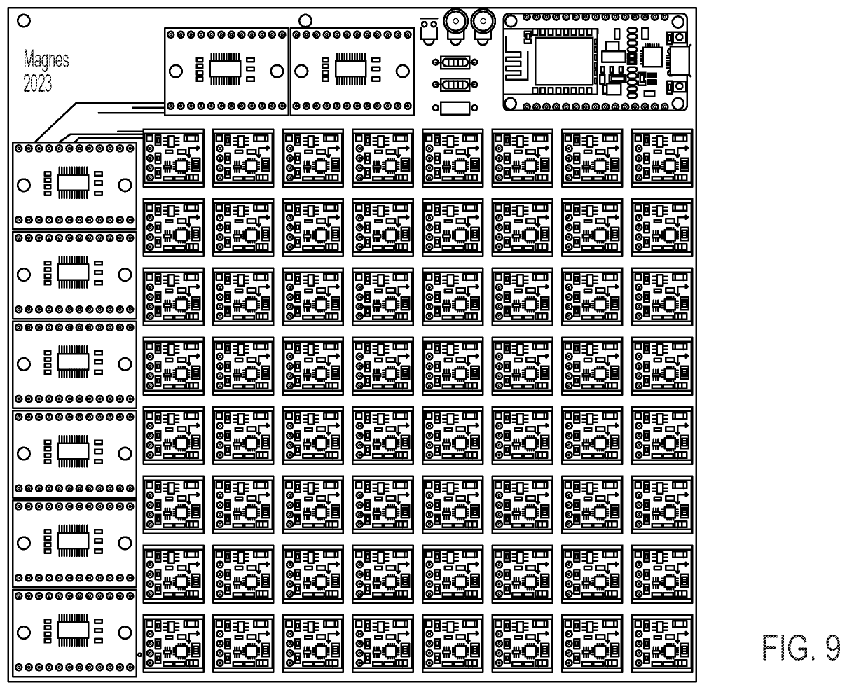

FIG. 8 shows a circuit board design of a system 800 according to examples of the present disclosure. The circuit board design of a system 800 comprises electrically coupling of one or more microcontroller 802, one or more magnetometers 804, one or more I2C multiplexers 806, one or more indicator LEDs 808. FIG. 9 shows another top view of FIG. 8.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. Moreover, the order in which the elements of the methods are illustrated and described may be re-arranged, and/or two or more elements may occur simultaneously. The embodiments were chosen and described in order to best explain the principles of the invention and its practical applications, to thereby enable others skilled in the art to best utilize the invention and various embodiments with various modifications as are suited to the particular use contemplated.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. Moreover, the order in which the elements of the methods are illustrated and described may be re-arranged, and/or two or more elements may occur simultaneously. The embodiments were chosen and described in order to best explain the principles of the invention and its practical applications, to thereby enable others skilled in the art to best utilize the invention and various embodiments with various modifications as are suited to the particular use contemplated.

Notwithstanding that the numerical ranges and parameters setting forth the broad scope of the embodiments are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 5. In certain cases, the numerical values as stated for the parameter can take on negative values. In this case, the example value of range stated as “less than 10” can assume negative values, e.g. −1, −2, −3, −10, −20, −30, etc.

The following embodiments are described for illustrative purposes only with reference to the Figures. Those of skill in the art will appreciate that the following description is exemplary in nature, and that various modifications to the parameters set forth herein could be made without departing from the scope of the present embodiments. It is intended that the specification and examples be considered as examples only. The various embodiments are not necessarily mutually exclusive, as some embodiments can be combined with one or more other embodiments to form new embodiments.

While the embodiments have been illustrated respect to one or more implementations, alterations and/or modifications can be made to the illustrated examples without departing from the spirit and scope of the appended claims. In addition, while a particular feature of the embodiments may have been disclosed with respect to only one of several implementations, such feature may be combined with one or more other features of the other implementations as may be desired and advantageous for any given or particular function.

Furthermore, to the extent that the terms “including”, “includes”, “having”, “has”, “with”, or variants thereof are used in either the detailed description and the claims, such terms are intended to be inclusive in a manner similar to the term “comprising.” As used herein, the phrase “one or more of”, for example, A, B, and C means any of the following: either A, B, or C alone; or combinations of two, such as A and B, B and C, and A and C; or combinations of A, B and C.

Other embodiments will be apparent to those skilled in the art from consideration of the specification and practice of the descriptions disclosed herein. It is intended that the specification and examples be considered as exemplary only, with a true scope and spirit of the embodiments being indicated by the following claims.

### BACKGROUND REFERENCES

Abbas, Mazen I, Maria Oliva-Hemker, Joon Choi, Michael Lustik, Mark A Gilger, R Adam Noel, Kathleen Schwarz, and Cade M Nylund. “Magnet ingestions in children presenting to US emergency departments, 2002-2011.” Journal of pediatric gastroenterology and nutrition 57, no. 1 (2013): 18-22.
Arshad, Muhammad, Sarah Masroor Jeelani, Areej Salim, and Bakhtawar Dilawar Hussain. “Multiple magnet ingestion leading to bowel perforation: A relatively sinister foreign body.” Cureus 11, no. 10 (2019).
Brown, Julie C, Jeffrey P Otjen, and George T Drugas. “Pediatric magnet ingestions: the dark side of the force.” The American Journal of Surgery 207, no. 5 (2014): 754-759.
Ciocirlan, Mihai. “Low-cost disposable endoscope: pros and cons.” Endoscopy international open 7, no. 09 (2019): E1184-E1186.
Dahshan, A. “Management of ingested foreign bodies in children.” The Journal of the Oklahoma State Medical Association 94, no. 6 (2001): 183-186.
Fryar, Cheryl D, Margaret D Carroll, Qiuping Gu, Joseph Afful, and Cynthia L Ogden. “Anthropometric Reference Data for Children and Adults: United States, 2015-2018.” Vital and Health Statistics 3, no. 46 (2021).
Hussain, Sunny Z, Athos Bousvaros, Mark Gilger, Petar Mamula, Sandeep Gupta, Robert Kramer, and R Adam Noel. “Management of ingested magnets in children.” Journal of pediatric gastroenterology and nutrition 55, no. 3 (2012): 239-242.
Kramer, Robert E, Diana G Lerner, Tom Lin, Michael Manfredi, Manoj Shah, Thomas C Stephen, Troy E Gibbons, Harpreet Pall, Ben Sahn, Mark McOmber, et al. “Management of ingested foreign bodies in children: a clinical report of the NASPGHAN Endoscopy Committee.” Journal of pediatric gastroenterology and nutrition 60, no. 4 (2015): 562-574.
Mandhan, Parkash, Muthana Alsalihi, Saleem Mammoo, and Mansour J Ali. “Troubling toys: rare-earth magnet ingestion in children causing bowel perforations.” Case reports in pediatrics 2014 (2014).
Middelberg, Leah K, Alexandra R Funk, Hannah L Hays, Lara B McKenzie, Bryan Rudolph, and Henry A Spiller. “Magnet Injuries in Children: An Analysis of the National Poison Data System from 2008 to 2019.” The Journal of Pediatrics, 2021.

Orsagh-Yentis, Danielle, Rebecca J McAdams, Kristin J Roberts, and Lara B McKenzie. “Foreign-body ingestions of young children treated in US emergency departments: 1995-2015.” Pediatrics 143, no. 5 (2019).
Waters, Alicia M, Daniel H Teitelbaum, Vivian Thorne, Athos Bousvaros, R Adam Noel, and Elizabeth A Beierle. “Surgical management and morbidity of pediatric magnet ingestions.” Journal of surgical research 199, no. 1 (2015): 137-140.
Webb, William A. “Management of foreign bodies of the upper gastrointestinal tract: update.” Gastrointestinal endoscopy 41, no. 1 (1995): 39-51.
Weiss, Audrey J, Lauren M Wier, Carol Stocks, and Janice Blanchard. “Overview of emergency department visits in the United States, 2011: statistical brief #174,” 2014.
Yuwono, Melawati, Alan Paschall, Jane L Ulrigg, Lisa Eagle, and Thomas Hurt. “Efficiency and Cost Effectiveness of Propofol Sedation in Endoscopic Foreign Body Removal in Pediatric Ambulatory Settings.” Gastrointestinal Endoscopy 69, no. 5 (2009): AB171.

1. A system for detecting a magnet, the system comprising:
an array of magnetometers;
one or more inter-integrated circuit (I2C) multiplexers electrically connected to the array of magnetometers; and
one or more wireless transceivers electrically connected to the one or more I2C multiplexers,
wherein the array of magnetometers detects a change in electrical resistance caused by a nearby magnetic object by measuring a strength of a magnetic field in three dimensions.

2. The system of claim 1, wherein each magnetometer of the array of magnetometers is connected to a I2C multiplexer of the one or more I2C multiplexers.

3. The system of claim 1, wherein the one or more I2C multiplexers are electrically connected to an I2C bus of the one or more wireless transceivers.

4. The system of claim 1, further comprising:
a computer,
wherein the one or more wireless transceivers transmit magnetic field data to the computer.

5. The system of claim 4, wherein the one or more wireless transceivers are electrically connected to the computer by a message queuing broker hosted on the computer.

6. The system of claim 5, wherein the message queuing broker is a message queuing telemetry transport broker.

7. The system of claim 4, wherein the computer is programed to localize the nearby magnetic object using both a magnetic heatmap and an x-ray.

8. The system of claim 7, wherein the computer is programmed to localize the nearby magnetic object by registering a magnetic heatmap image and an x-ray image to create an overlay of the magnetic heatmap image and the x-ray image that allows a clinician to identify magnetic objects that appear radiopaque on the x-ray image.

9. The system of claim 8, wherein the registration of the magnetic heatmap image and the x-ray image comprises of performing an affine transformation between a plurality of magnetic markers in both the magnetic heatmap image and the x-ray image.

10. The system of claim 4, wherein the computer is programmed to generate an overlay of a magnetic heatmap and an x-ray image.

11. The system of claim 4, wherein the computer is programmed to generate a user interface for manipulating images of a magnetic heatmap, an x-ray image, or an overlay of the magnetic heatmap and the x-ray image.

12.-14. (canceled)

15. A system for detecting a magnet, the system comprising:
a handheld device comprising an array of magnetometers, one or more inter-integrated circuit (I2C) multiplexers electrically connected to the array of magnetometers, one or more wireless transceivers electrically connected to the one or more I2C multiplexers, wherein the array of magnetometers detects a change in electrical resistance caused by a nearby magnetic object by measuring a strength of a magnetic field in three dimensions; and
a computer programmed to generate a user interface for displaying a magnetic heat map image, an x-ray image, and an overlay of the magnetic heatmap image and the x-ray image, wherein the one or more wireless transceivers transmit magnetic field data to the computer.

16. The system of claim 15, wherein each magnetometer of the array of magnetometers is connected to a I2C multiplexer of the one or more I2C multiplexers.

17. The system of claim 15, wherein the one or more I2C multiplexers are electrically connected to an I2C bus of the one or more wireless transceivers.

18. The system of claim 17, wherein the one or more wireless transceivers are electrically connected to the computer by a message queuing broker hosted on the computer.

19. The system of claim 18, wherein the message queuing broker is a message queuing telemetry transport broker.
